(19) United States
(12) Patent Application Publication
(10) Pub. No.: US 2014/0243936 A1
(43) Pub. Date: Aug. 28, 2014

(54) BEAM THERAPY APPARATUS AND METHOD FOR CONTROLLING SAME

(71) Applicant: Lutronic Corporation, Gyeonggi-do (KR)

(72) Inventor: Tae Ho Ha, Gyeonggi-do (KR)

(73) Assignee: LUTRONIC CORPORATION, Gyeonggi-do (KR)

(21) Appl. No.: 14/348,496

(22) PCT Filed: Sep. 28, 2012
(86) PCT No.: PCT/KR2012/007939

§ 371 Date: Mar. 28, 2014

(30) Foreign Application Priority Data
Sep. 30, 2011 (KR) 10-2011-0099895
Oct. 31, 2011 (KR) 10-2011-0112345

(51) Int. Cl. A61N 5/06

(52) U.S. Cl. CPC A61N 5/0613
USPC 607/90

(57) ABSTRACT

The present invention relates to a beam therapy apparatus comprising: a main body; a beam generating unit accommodated in an interior of the main body to generate a therapy beam for treating lesions in a patient; a beam delivery unit accommodated in the interior of the main body to guide the beam generated by the beam generating unit from the main body to the lesions in the patient; a pattern forming unit which corresponds to the lesion area in the patient and forms a pattern constituted by a plurality of divided cells to which the therapy beam from the beam delivery unit is radiated; and a control unit which controls an operation of the beam delivery unit such that the therapy beam may be radiated sequentially or at random to the cells based on the shape of the pattern formed by the pattern forming unit.

### TECHNICAL FIELD

The present invention relates to a beam therapy apparatus and a method for controlling the same, and more particularly, to a beam therapy apparatus for irradiating a therapeutic beam in a pattern to a lesion of a patient, and a method for controlling the same.

### BACKGROUND ART

A beam therapy apparatus is a medical apparatus that irradiates a beam to lesion of a patient to treat it. In particular, among beam therapy apparatuses, a treatment apparatus using a laser irradiates a therapeutic laser having a predetermined wavelength to a lesion of a skin or an eyeball of a human body for a predetermined irradiation time to treat the lesion region.

Meanwhile, a related art treatment apparatus is disclosed in U.S. Pat. Registration No. 5,549,596 entitled “SELECTIVE LASER TARGETING OF PIGMENTED OCULAR CELLS”. This document discloses a technique of shortening a treatment time by irradiating a plurality of lasers in a pattern of a predetermined region to a lesion of a patient.

However, in the treatment apparatus disclosed in the related art document, since a plurality of lasers are irradiated to a pattern of a predetermined region to shorten a treatment time, cells of a patient, in particular, normal cells of a patient, may also be destructed due to a degradation phenomenon.

### DISCLOSURE

### Technical Problem

An aspect of the present invention provides a beam therapy apparatus having a structure improved to hinder destruction of human cells due to a degradation in irradiating a therapeutic beam in a pattern to a lesion region of a patient.

### Technical Solution

According to an aspect of the present invention, there is provided a beam therapy apparatus including: a main body; a beam generating unit accommodated in the main body and configured to generate a therapeutic beam to treat a lesion of a patient; a beam delivery unit accommodated in the main body and configured to deliver the beam generated by the beam generating unit to the lesion of the patient from the main body; a pattern forming unit configured to form a pattern corresponding to the lesion region of the patient and composes of a plurality of divided cells to which the therapeutic beam is irradiated from the beam delivery unit; and a controller configured to control the beam delivery unit to irradiate the therapeutic beam sequentially or randomly to the cells based on a shape of the pattern formed by the pattern forming unit.

The beam delivery unit may include: a beam expanding unit configured to expand the therapeutic beam generated by the beam generating unit; a lens unit configured to irradiate the expanded therapeutic beam from the beam expanding unit to the outside of the main body; and a scanner disposed between the beam expanding unit and the lens unit to adjust a path of the therapeutic beam to be irradiated according to the pattern formed by the pattern forming unit.

The scanner may include: a first induction unit rotated based on an axial line of an X axis to induce the therapeutic beam provided from the beam expanding unit in a horizontal direction of a rotation axial line based on the X axis; and a second induction unit rotated based on an axial line of a Y axis perpendicular to the rotation axial line of the first induction unit to induce the therapeutic beam induced by the first induction unit, in a horizontal direction of the rotation axial line based on the Y axis.

The pattern may include any one of circular, oval, polygonal, and curved closed loop shapes.

The cells may be regions obtained by dividing the interior of the pattern into a plurality of areas each having the same size.

The pattern forming unit may further form auxiliary cells obtained by dividing the interior of each cell into a plurality of areas each having the same size.

The controller may control the beam delivery unit to irradiate the therapeutic beam sequentially or randomly to at least every other cell of the plurality of cells.

The controller may control the beam delivery unit to irradiate the therapeutic beam sequentially or randomly to at least every other auxiliary cell of the plurality of auxiliary cells.

According to another aspect of the present invention, there is provided a control method of a beam therapy apparatus, including: (a) forming a pattern including a plurality of divided cells, corresponding to a lesion region of a patient, to which a therapeutic beam is irradiated; (b) generating a therapeutic beam irradiated to the pattern; (c) irradiating the therapeutic beam to the lesion region of the patient; and (d) adjusting an irradiated position of the therapeutic beam irradiated in operation (c) based on a shape of the pattern formed in operation (a), and sequentially or randomly irradiating the therapeutic beam to the cells.

The cells of the pattern formed in operation (a) may be regions obtained by dividing the interior of the pattern into a plurality of areas each having the same size.

In operation (d), the therapeutic beam may be irradiated sequentially at every other cell of the plurality of cells, or randomly.

In operation (a), auxiliary cells obtained by dividing the interior of each cell into a plurality of areas each having the same size may be further formed.

In operation (d), when the auxiliary cells are formed, an irradiated position of the therapeutic beam irradiated in operation (c) may be adjusted to irradiate the therapeutic beam to the auxiliary cells sequentially or randomly.

Also, preferably, when the therapeutic beam is irradiated to the auxiliary cells, the therapeutic beam may be irradiated sequentially or randomly at every other auxiliary cell of the plurality of auxiliary cells.

Specific matters of other embodiments are included in the detailed description and drawings.

### Advantageous Effects

In the beam therapy apparatus 100 and the method for controlling the same according to the present invention, by irradiating a therapeutic beam having a pattern corresponding to a lesion region of a patient, time for a treatment may be shortened, and by discontinuously irradiating a therapeutic beam to each of a plurality of cells obtained by dividing a pattern or to each of auxiliary cells obtained by dividing a cell, a degradation destructing human cells may be prevented.

### DESCRIPTION OF DRAWINGS

FIG. 1 is a block diagram illustrating a beam therapy apparatus 10 according to an embodiment of the present invention;

FIG. 2 is a perspective view illustrating a configuration of a beam delivery unit illustrated in FIG. 1;

FIG. 3 is a view illustrating formation of a pattern and irradiation of a therapeutic beam of the beam therapy apparatus 10 according to a first embodiment of the present invention;

FIG. 4 is a view illustrating formation of a different pattern and irradiation of a therapeutic beam of the beam therapy apparatus 10 according to a first embodiment of the present invention;

FIG. 5 is a flow chart illustrating an operation of the beam therapy apparatus 10 according to the first embodiment of the present invention;

FIG. 6 is a view illustrating formation of a pattern and irradiation of a therapeutic beam of the beam therapy apparatus 10 according to a second embodiment of the present invention; and

FIG. 7 is a flow chart illustrating an operation of the beam therapy apparatus 10 according to the second embodiment of the present invention.

### BEST MODES

Hereinafter, a beam therapy apparatus 10 and a method for controlling the same according to embodiments of the present invention will be described in detail with reference to the accompanying drawings.

To begin with, it should be appreciated that the beam therapy apparatus 10 and a method for controlling the same according to first and second embodiments of the present invention are described for the purpose of ophthalmic treatment but those may also be used for a treatment of skin.

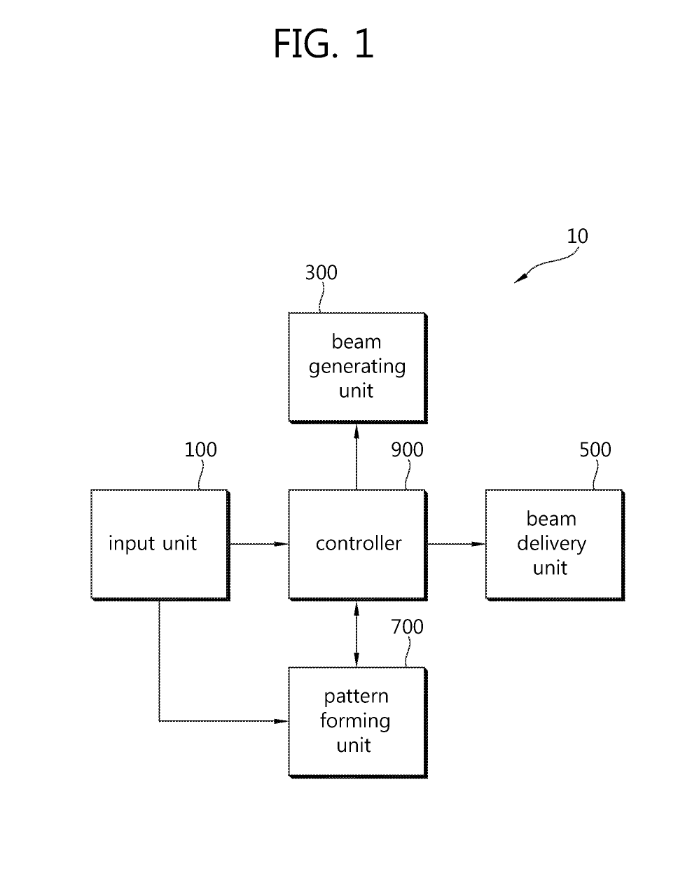

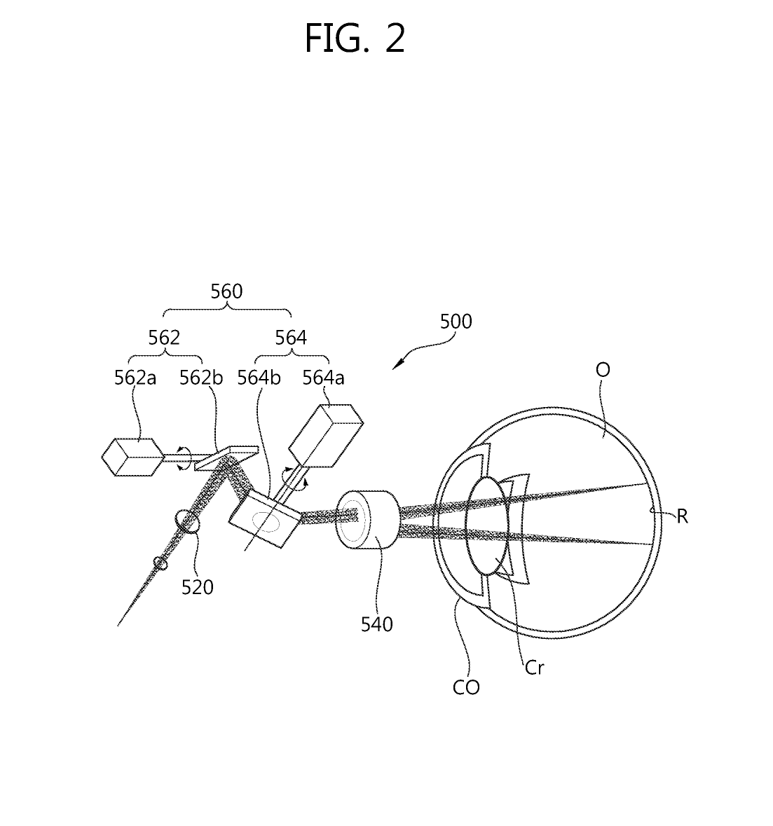

FIG. 1 is a block diagram illustrating a beam therapy apparatus 10 according to an embodiment of the present invention, and FIG. 2 is a perspective view illustrating a configuration of a beam delivery unit illustrated in FIG. 1.

As illustrated in FIGS. 1 and 2, the beam therapy apparatus 10 according to the embodiment of the present invention includes an input unit 100, a beam generating unit 300, a beam delivery unit 500, a pattern forming unit 700, and a controller 900. Also, the beam therapy apparatus 10 according to the embodiment of the present invention further includes a main body (not shown) in which the input unit 100, the beam generating unit 300, the beam delivery unit 500, the pattern forming unit 700, and the controller 900 are installed or accommodated.

The main body includes an examination unit (not shown) forming the exterior of the beam therapy apparatus 10 and having a high magnification ratio such as a microscope to examine eye conditions of a patient before treating a lesion of the patient's eyes. The input unit 100 is disposed outside of the main body to apply an input signal, and the beam generating unit 300, the beam delivery unit 500, the pattern forming unit 700, and the controller 900 are accommodated within the main body.

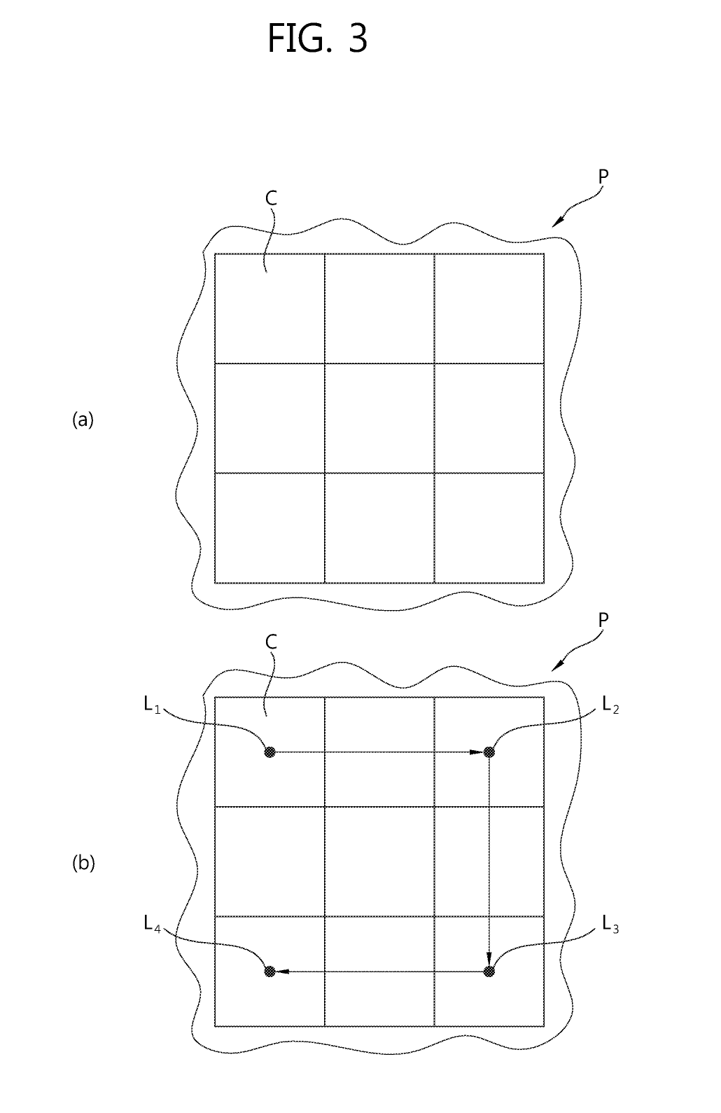

The input unit 100 disposed outside of the main body applies an input signal for operating the beam generating unit 300 and the beam delivery unit 500. The input unit 100 may be configured as a joystick or a pedal. The input unit 100 applies an input signal for operating the beam generating unit 300 and the beam delivery unit 500 according to an input operation of an operator. Here, the input unit 100 may apply a signal to the pattern forming unit 700 to allow patterns P and P corresponding to the lesion of the patient, among various patterns P and P (please refer to FIGS. 3, 4, and 6) stored in advance, to be selected.

The beam generating unit 300 generates a therapeutic beam based on the input signal applied from the input unit 100. As the beam generating unit 300 according to the embodiment of the present invention, a laser diode generating a layer may be used. Of course, a therapeutic beam generated by the beam generating unit 300 may vary according to types of light sources (not shown). A therapeutic beam generated by the beam generating unit 300 has a wavelength ranging from 1064 nm to 532 nm. However, besides the wavelength ranging from 1064 nm to 532 nm, the therapeutic beam generated by the beam generating unit 300 may also have a wavelength exceeding 1064 nm or less than 532 nm depending on a therapy purpose or a therapy target lesion.

The beam delivery unit 500 includes a beam expanding unit 520, a lens unit 540, and a scanner 560 according to the embodiment of the present invention. The beam delivery unit 500 delivers the therapeutic beam generated by the beam generating unit 300 to the lesion of the patient. Here, the beam delivery unit 500 adjusts an irradiated position of the therapeutic beam to correspond to the patterns P and P.

The beam expanding unit 520 is disposed to be adjacent to the beam generating unit 300 to expand a therapeutic beam. Namely, the beam expanding unit 620 expands the therapeutic beam generated by the beam generating unit 300 and guides the expanded beam to the lens unit 540. The lens unit 540 irradiates the expanded therapeutic beam from the beam expanding unit 520 to the outside of the main body.

The scanner 560 is disposed between the beam expanding unit 520 and the lens unit 540 and adjusts a path of the therapeutic beam to be irradiated according to the patterns P and P formed by the pattern forming unit 700. The scanner 560 adjusts a path of the expanded therapeutic beam from the bean expanding unit 520 and guides the same to the lens unit 540. According to the embodiment of the present invention, the scanner 560 includes a first induction unit 562 and a second induction unit 564.

The first induction unit 562 includes a first driving unit 562a and a first mirror unit 562b. The first induction unit 562 is rotated based on an axial line of an X axis to induce the therapeutic beam provided from the beam expanding unit 520 in a horizontal direction of the rotation axial line based on the X axis. Here, the first driving unit 562a is connected to the first mirror unit 562b to generate driving force rotating the first mirror unit 562b. The first mirror unit 562b is rotated along the rotation axial line based on the X axis according to driving force provided from the first driving unit 562a to induce the therapeutic beam to the second induction unit 564.

The second induction unit 564 includes a second driving unit 564a and a second mirror unit 564b. The second induction unit 564 is rotated based on an axial line of a Y axis perpendicular to the rotation axial line of the first induction unit 562 to induce the therapeutic beam induced by the first induction unit 562 in a horizontal direction of the rotation axial line based on the Y axis. Here, the second driving unit 564a is connected to the second mirror unit 564b to generate driving force rotating the second mirror unit 564b. The second mirror unit 564b is rotated along the rotation axial line based on the Y axis according to the driving force provided from the second driving unit 564a to induce the therapeutic beam to the lens unit 540.

### First Embodiment

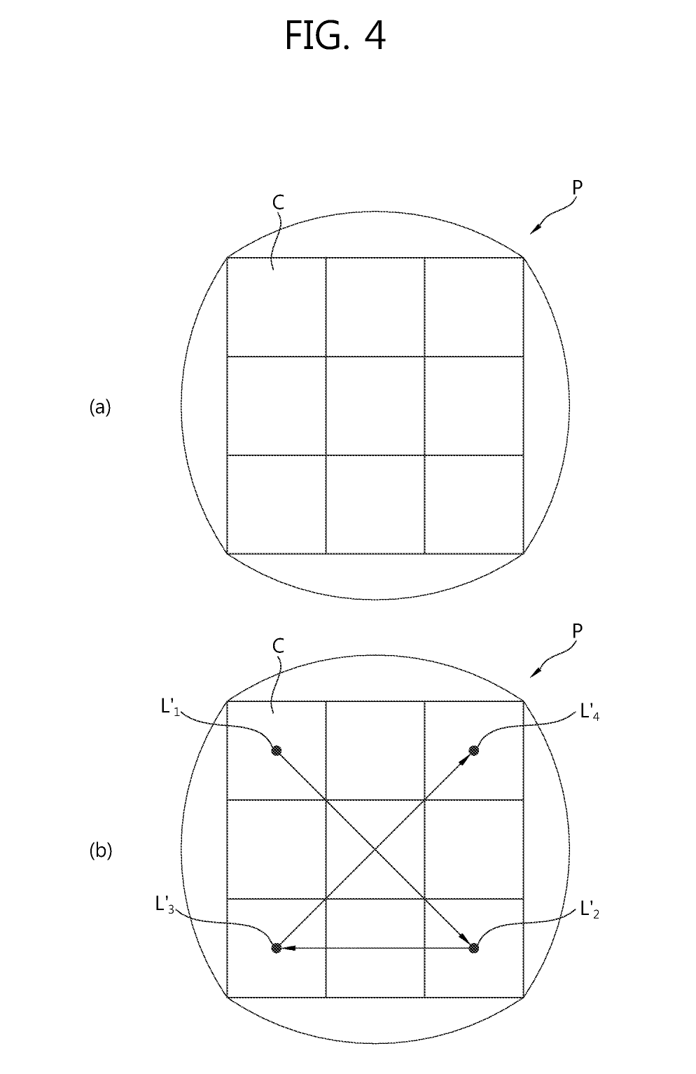

FIG. 3 is a view illustrating formation of a pattern and irradiation of a therapeutic beam of the beam therapy apparatus 10 according to a first embodiment of the present invention, and FIG. 4 is a view illustrating formation of a different pattern and irradiation of a therapeutic beam of the beam therapy apparatus 10 according to a first embodiment of the present invention.

As illustrated in FIGS. 3 and 4, the pattern forming unit 700 of the beam therapy apparatus 10 according to the first embodiment of the present invention forms a pattern P that corresponds to a lesion region of a patient and to which the therapeutic beam is irradiated from the beam delivery unit 500.

As illustrated in (a) of FIG. 3, the pattern forming unit 700 forms the pattern P having cells C each having the same area within a curved closed loop. A therapeutic beam is sequentially irradiated to the pattern P composed of the plurality of cells C under the control of the controller 900 as illustrated in (b) of FIG. 3. Here, in FIG. 3, it is illustrated that the therapeutic beam is sequentially irradiated, but the therapeutic beam may also be randomly irradiated.

Also, as illustrated in (a) of FIG. 4, a pattern P having cells each having the same area may be formed within a circle. As illustrated in (b) of FIG. 4, the therapeutic beam is sequentially irradiated to the pattern P composed of the plurality of cells C under the control of the controller 900. However, FIG. 4 merely illustrates an example and the therapeutic beam may be randomly irradiated. Namely, the pattern forming unit 700 may selectively form a pattern P having any shape among a circular, oval, polygonal, and curved closed loop shapes. The pattern P may be selected by an input signal of the input unit 100 or any one of previously stored patterns P may be automatically selected by the controller 900 according to a size of a lesion of a patient.

Adjustment of a irradiated position to which the therapeutic beam is irradiated under the control of the controller 900 will be described in conjunction with the controller 900 hereinafter.

The controller 900 controls the beam delivery unit 500 to irradiate the therapeutic beam to the cell C sequentially or randomly based on the shape of the pattern P formed by the pattern forming unit 700. Namely, the controller controls an operation of the beam delivery unit 500 in order to adjust a irradiated position of the therapeutic beam irradiated to retina R through cornea CO and crystalline lens Cr of the eyeball O.

The controller 900 controls the beam delivery unit 500 to irradiate the therapeutic beam sequentially to every second one of the plurality of cells C of the pattern P, namely, alternately, or to irradiate the therapeutic beam to the plurality of cells C of the pattern P randomly. For example, as illustrated in (b) of FIG. 3, the controller 900 may control the beam delivery unit 500 to irradiate the therapeutic beam to every second one of the cells C, starting from the uppermost cell C in the left, in a clockwise direction of L₁, L₂, L₃, and L₄. Meanwhile, as illustrated in (b) of FIG. 4, the controller 900 may control the beam delivery unit 500 to irradiate the therapeutic beam to every second one of the cells, starting from the uppermost cell C in the left, in a diagonal direction of L₁′, L₂′, L₃′, and L₄′.

In this manner, since the controller 90 irradiates the therapeutic beam to at least every second one of the cells C, rather than continuously to the contiguous cells C, a degradation due to the irradiation of the therapeutic beam may be prevented.

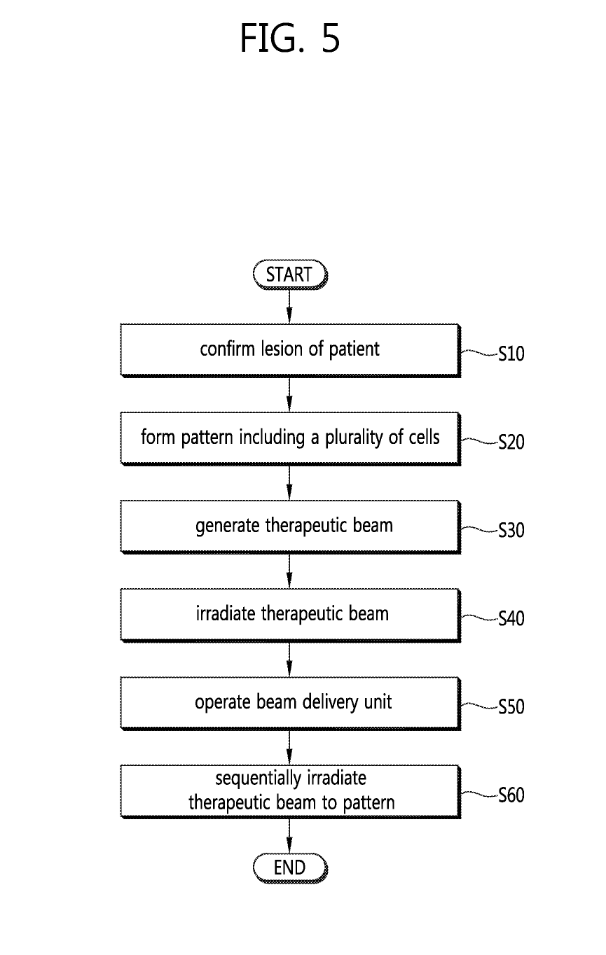

FIG. 5 is a flow chart illustrating an operation of the beam therapy apparatus 10 according to the first embodiment of the present invention.

A control method of the beam therapy apparatus 10 according to the first embodiment of the present invention having the aforementioned configuration will be described hereinafter.

First, a lesion generated in the eyeball of a patient is checked through the examination unit provided in the main body (S10). A pattern P corresponding to the lesion of the patient is formed (S20). In this case, the pattern P includes a plurality of cells C each having the same area therein. An input signal is applied to the input unit 100 to generate a therapeutic beam (S30).

The therapeutic beam generated by the beam generating unit 300 is irradiated (S40). Here, an operation of the beam delivery unit 500 irradiating the beam generated by the beam generating unit 300 to the lesion of the patient is controlled (S50). The beam delivery unit 500 is operated to irradiate a therapeutic beam to every noncontiguous cell C of the plurality of cells C of the pattern P under the control of the controller 900.

The therapeutic beam is irradiated to each cell C such that it is sequentially irradiated to every second one of the plurality of cells C of the pattern P by operating the beam delivery unit 500 (S60). Here, the therapeutic beam irradiated to each cell C is sequentially displayed for the description purpose, but substantially, an irradiation time is extremely short, and thus, it may be seen that the therapeutic beam is irradiated to the entire cell C region of the pattern P at a time. In this manner, since the therapeutic beam is irradiated to every noncontiguous cell C, a degradation of human cells may be advantageously reduced.

### Second Embodiment

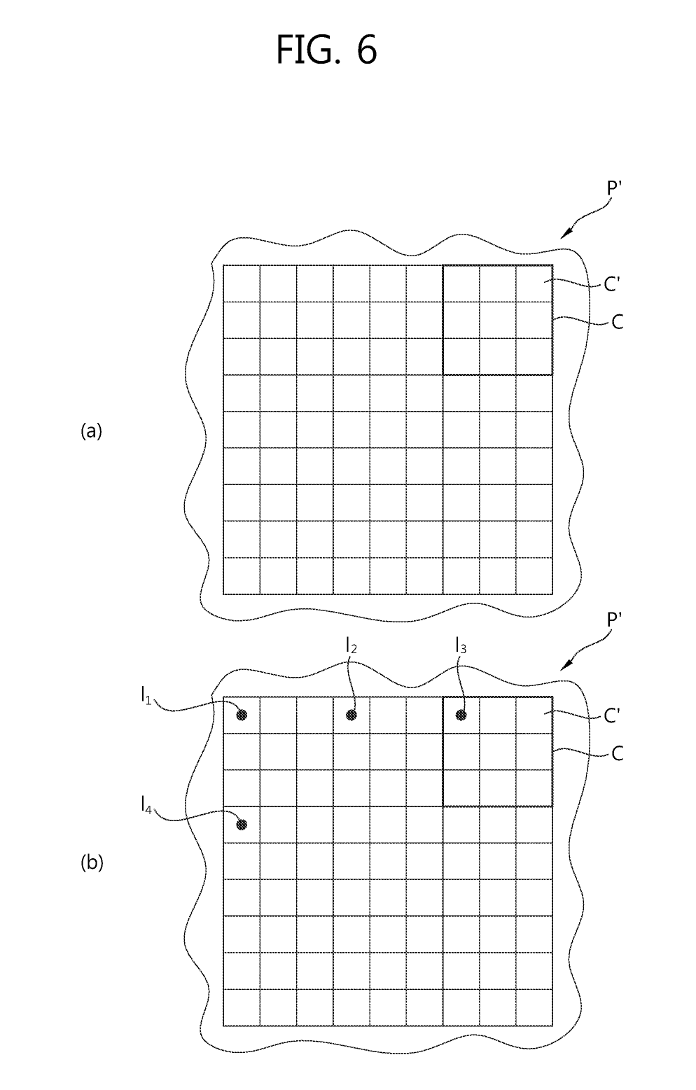

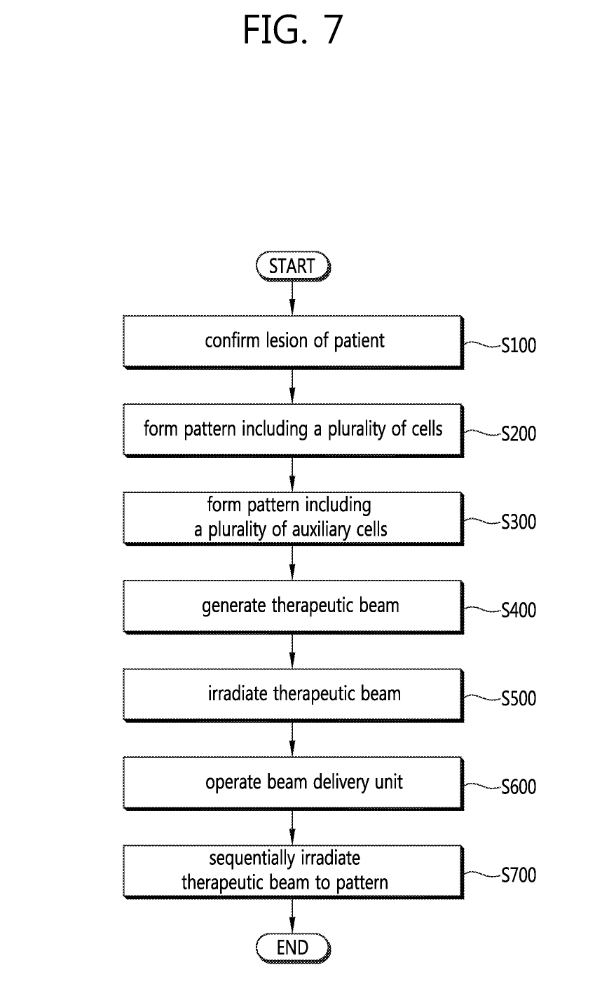

FIG. 6 is a view illustrating formation of a pattern and irradiation of a therapeutic beam of the beam therapy apparatus 10 according to a second embodiment of the present invention, and FIG. 7 is a flow chart illustrating an operation of the beam therapy apparatus 10 according to the second embodiment of the present invention.

As illustrated in FIGS. 6 and 7, the beam therapy apparatus 10 according to the second embodiment of the present invention is identical to that of the first embodiment, except that auxiliary cells C′ are formed by dividing the interior of cells C of a pattern P′ formed by the pattern forming unit 700.

Namely, in the beam therapy apparatus 10 according to the second embodiment of the present invention, the interior of each cell C of the pattern P includes a plurality of auxiliary cells C each having the same area to allow a large amount of therapeutic beams to be irradiated. For example, as illustrated in (b) of FIG. 6, a therapeutic beam is irradiated to the auxiliary cells C, starting from the uppermost portion of each cell C in the left, in order of I₁, I₂, I₃, and I₄.

Here, the beam therapy apparatus 10 according to the second embodiment of the present invention sequentially irradiates the therapeutic beam to the auxiliary cells C at the same position of each cell C, but it may also irradiate the therapeutic beam in various other manners, for example, randomly, such as in order of the first cell C, the third cell C, and the fifth cell C. Of course, the sequential irradiation of the therapeutic beam needs to be performed at every predetermined interval in order to prevent a degradation of human cells.

Also, the therapeutic beam is irradiated to every second auxiliary cells C of each cell, but it may also be irradiated to the auxiliary cells by twos at a time such that the therapeutic beam is irradiated to two auxiliary cells C with the exception of one auxiliary cell therebetween. Namely, an irradiated position or order of the therapeutic beam irradiated by the beam therapy apparatus 10 according to the embodiment of the present invention may be conducted according to a pre-set pattern P′ value in various manners such that the cells of the patient are not degraded. As described above, the therapeutic beam may be sequentially irradiated to every auxiliary cell C′ at the same position of each cell C′, sequentially irradiated to every auxiliary cell C′ at different positions of each cell C, or sequentially irradiated to the auxiliary cells C′ of each cell by twos with the exception of one therebetween. The irradiation of the therapeutic beam may be made in various combinations within a range in which a degradation of human cells is prevented.

With the foregoing configuration, a control method of the beam therapy apparatus 10 according to the second embodiment of the present invention will be described.

First, a lesion generated in the eyeball O of a patient is checked through the examination unit provided in the main body (S100). A pattern P composed of a plurality of cells C each having the same area, which corresponds to the lesion of the patient, is formed (S200). Each of the plurality of cells C has a plurality of auxiliary cells C each having the same area therein (S300).

An input signal is applied to the input unit 100 to generate a therapeutic beam (S400). The therapeutic beam generated by the beam generating unit 300 is irradiated (S500). Here, an operation of the beam delivery unit 500 that irradiates the therapeutic beam generated by the beam generating unit 300 to the lesion of the patient is controlled (S600). Then, the beam delivery unit 500 is operated to irradiate a therapeutic beam to every noncontiguous cells C of the plurality of cells C of the pattern P′ under the control of the controller 900.

The therapeutic beam is sequentially irradiated to every second auxiliary cell C′ of the plurality of cells C′ formed in each cell C of the pattern P′ by the operation of the beam delivery unit 500, thus irradiating the therapeutic beam to each auxiliary cell C′ (S700).

Thus, since the therapeutic beam is irradiated based on the pattern corresponding to the lesion region of the patient, a time for a therapy may be shortened and, since the therapeutic beam is sequentially irradiated to the regions obtained by dividing a pattern, a degradation of damaging human cells may be prevented.

The foregoing embodiments and advantages are merely exemplary and are not to be considered as limiting the present disclosure. It will be apparent to those skilled in the art that modifications and variations can be made without departing from the spirit and scope of the invention. This description is intended to be illustrative, and not to limit the scope of the claims. Also, although an embodiment has not been described in the above disclosure, it should be extensively construed within the scope of the technical concept defined in the claims. And, various changes and modifications that fall within the scope of the claims, or equivalents of such scope are therefore intended to be embraced by the appended claims

1. A beam therapy apparatus comprising:
a main body;
a beam generating unit accommodated in the main body and configured to generate a therapeutic beam to treat a lesion of a patient;
a beam delivery unit accommodated in the main body and configured to deliver the beam generated by the beam generating unit to the lesion of the patient from the main body;
a pattern forming unit configured to form a pattern corresponding to the lesion region of the patient and composed of a plurality of divided cells to which the therapeutic beam is irradiated from the beam delivery unit; and
a controller controlling the beam delivery unit to irradiate the therapeutic beam sequentially or randomly to the cells based on a shape of the pattern formed by the pattern forming unit.

2. The beam therapy apparatus of claim 1, wherein the beam delivery unit comprises:
a beam expanding unit configured to expand the therapeutic beam generated by the beam generating unit;
a lens unit configured to irradiate the expanded therapeutic beam from the beam expanding unit to the outside of the main body; and
a scanner disposed between the beam expanding unit and the lens unit to adjust a path of the therapeutic beam to be irradiated according to the pattern formed by the pattern forming unit.

3. The beam therapy apparatus of claim 2, wherein the scanner comprises:
a first induction unit rotated based on an axial line of an X axis to induce the therapeutic beam provided from the beam expanding unit in a horizontal direction of a rotation axial line based on the X axis; and
a second induction unit rotated based on an axial line of a Y axis perpendicular to the rotation axial line of the first induction unit to induce the therapeutic beam induced by the first induction unit, in a horizontal direction of the rotation axial line based on the Y axis.

4. The beam therapy apparatus of claims 1 to 3, wherein the pattern includes any one of circular, oval, polygonal, and curved closed loop shapes.

5. The beam therapy apparatus of claim 4, wherein the cells are regions obtained by dividing the interior of the pattern into a plurality of areas each having the same size.

6. The beam therapy apparatus of claim 5, wherein the pattern forming unit further forms auxiliary cells obtained by dividing the interior of each cell into a plurality of areas each having the same size.

7. The beam therapy apparatus of claim 4, wherein the controller controls the beam delivery unit to irradiate the therapeutic beam sequentially or randomly to at least every other cell of the plurality of cells.

8. The beam therapy apparatus of claim 6, wherein the controller controls the beam delivery unit to irradiate the therapeutic beam sequentially or randomly to at least every other auxiliary cell of the plurality of auxiliary cells.

9. A control method of a beam therapy apparatus, the control method comprising:
(a) forming a pattern including a plurality of divided cells, corresponding to a lesion region of a patient, to which a therapeutic beam is irradiated;
(b) generating a therapeutic beam irradiated to the pattern;
(c) irradiating the therapeutic beam to the lesion region of the patient; and
(d) adjusting an irradiated position of the therapeutic beam irradiated in operation (c) based on a shape of the pattern formed in operation (a), and sequentially or randomly irradiating the therapeutic beam to the cells.

10. The control method of claim 9, wherein the cells of the pattern formed in operation (a) are regions obtained by dividing the interior of the pattern into a plurality of areas each having the same size.

11. The control method of claim 9, wherein, in operation (d), the therapeutic beam is irradiated sequentially at every other cell of the plurality of cells, or randomly.

12. The control method of claim 10, wherein, in operation (a), auxiliary cells are further formed by dividing the interior of each cell into a plurality of areas each having the same size

13. The control method of claim 12, wherein, in operation (d), when the auxiliary cells are formed, an irradiated position of the therapeutic beam irradiated in operation (c) is adjusted to irradiate the therapeutic beam to the auxiliary cells sequentially or randomly.

14. The control method of claim 13, wherein when the therapeutic beam is irradiated to the auxiliary cells, the therapeutic beam is irradiated sequentially or randomly at every other auxiliary cell of the plurality of auxiliary cells.
